(19) United States
(12) Patent Application Publication
(10) Pub. No.: US 2026/0346072 A1
(43) Pub. Date: Nov. 5, 2026

(54) BIOTECHNOLOGICAL COMPOSITION, PROCESS AND USE TO CONTROL SILICA

(71) Applicant: DOMOLIF SPA, Antofagasta (CL)

(72) Inventors: Pamela Chávez Crooker, Antofagasta (CL); Andrea Eusebia Del Milagro Contreras Vera, Antofagasta (CL); Johanna Del Rosario Obreque Contreras, Antofagasta (CL)

(73) Assignee: DOMOLIF SPA, Antofagasta (CL)

(21) Appl. No.: 18/997,480

(22) PCT Filed: Jul. 21, 2023
(86) PCT No.: PCT/CL2023/050059

§ 371 Date: Jan. 21, 2025

(30) Foreign Application Priority Data
Jul. 21, 2022 (CL) 202201978
Jul. 20, 2023 (CL) 202302123

(51) Int. Cl. A62D 3/02; A62D 101/40; C12N 1/20; C12N 15/75; C12P 3/00; C12R 1/07

(52) U.S. Cl. CPC A62D 3/02; C12N 1/20; C12N 15/75; A62D 2101/40; C12P 3/00; C12R 2001/07

(57) ABSTRACT

The present invention relates to a biotechnological composition, a process and the use thereof to immobilize and reduce, in situ, silica present in particulate material suspended in the atmosphere.

### FIELD OF THE INVENTION

The invention relates to a biotechnological composition, a process, and its use for containing and reducing—in situ—silica in suspended particulate matter found in the environment.

### Background of the Invention

Silicosis is an occupational disease caused by the inhalation of crystalline SiO2 (silica), especially in mining operations. This disease is one of several pulmonary complications associated with toxic exposures in the workplace and is an irreversible lung pathology that can lead to death. Measures implemented by health authorities have not been successful and there is no technological innovation to solve the problem at the source.

There are currently no dust suppressants aimed to modify chemical and structural composition of silica dioxide—in situ—.

The state-of-the-art reports there is a natural process by which silica dioxide can be transformed in its structure and chemistry. This process is a silica leaching mediated by a bioremediation enzyme where silicification occurs due to the action of the enzyme by using a biochemical reaction aimed to form gehlenite (Ca2Al2SiO7) in the presence of Calcium and Aluminum.

For example, the non-patent paper titled “Development of an improved E. coli bacterial strain for green and sustainable concrete technology”, Sarkar et al., 2015, describes the increase of compressive strength, stiffness and longevity of concrete structures with the help of bioengineering techniques in the field of construction. In this paper, a genetic manipulation technique was used to develop new characteristics within E. coli bacteria (JMJ107) by transferring the silica-leaching gene from BKH2 bacteria. The transformed bacteria were incorporated into cementitious mortar to observe their usefulness in terms of compressive strength and durability of the modified material.

In addition, the non-patent paper entitled “Genetically-enriched microbe-facilitated self-healing concrete—A sustainable material for a new generation of construction technology” Sarkar et al., 2015, describes the development of self-healing property of cementitious material in construction technology by means of a biomineralization process produced by bacteria, where the application of a genetically modified bacteria for green engineering and a true self-healing process over a prolonged period will create a new hope for sustainable concrete technology. In this work, the biosilicification gene of BKH2 has been transformed into a spore-forming bacterium Bacillus subtilis. Then the genetically modified bacterium has been used in ordinary Portland cement-based concrete material for crack healing during long periods, aimed to develop a self-repairing new material.

Additionally, patent CL 51248 for dust control is known in the state-of-the-art, referring to a composition for reducing particulate matter in suspension in air or a liquid comprising a source of exopolysaccharides (EPS), a strain of microorganisms with ureolytic activity and culture medium, apart from a method for reducing particulate matter in suspension in air or a liquid, involving the application of said composition.

Patent CL 50541 also describes a method for reducing suspended particulate matter in air or water.

However, the technical issue of these documents focuses on construction, more specifically on improving the strength and durability of concrete, or on methods to control suspended dust. No mention is made about containing and removing silica present in suspended particulate matter in soil, tailings or any other silica-rich material in mining operations environment, aimed to reducing the risk of silicosis caused by inhalation of crystalline SiO2.

Based on the above, this patent application describes a composition, a process and its use to control particulate matter. In addition, it removes silica present in suspended particulate matter in the environment, such as mining operations, especially in mine pits, crushing areas and loading faces, as well as in tailing dams.

The composition comprises a genetically modified bacterium with a natural biocementation activity, thus forming a high concentration of carbonate in its environment. It was cloned with a gene that synthesizes an enzyme capable of transforming silica dioxide by means of a biological activity known as silicification, where silica is released to bind in a covalent manner to carbonates formed by biocementation thus to form new, larger silica crystals, aimed to reducing the risk of silicosis when inhaling crystalline SiO2. Biocementation and silicification are combined into an organism aimed to eliminate SiO2 by transforming it into new, larger crystals.

Therefore, this invention proposes a composition consisting of a bacterium with dual activity: i) biocementation: ability to form calcite and ii) silicification, by modifying silica to gehlenite and/or other silica minerals of significantly larger size, wherein said bacterium contains the gene inserted in its genome and constitutively expresses the enzyme.

Among the advantages of this invention, the following ones can be highlighted:
100% organic and environmentally friendly product.
It removes breathable silica, irreversibly eliminating the source of SiO₂ from the soil.
It is an inexpensive and easy to scale technology.
Allows for the installation of capabilities to address other problems, such as asbestos and tailings contamination.
The technology is transferable to other industries, such as construction, so its impact ranges from regional to national.
Allows to tackle the source of the silica problem and also to control particulate matter.
The biotechnological composition comprises environmentally friendly and natural bacteria. It does not generate toxic waste and accelerates a natural process of silica transformation into a stable and irreversible mineral, gehlenite and other minerals containing silica of non-breathable size.

### DESCRIPTION OF THE FIGURES

FIG. 1 describes the strength results of controlled test specimens, where the fine particulate matter (chusca) is soil with a granulometry 90%<0.075 (mm).

FIG. 2a describes a control specimen subjected to compression. FIG. 2a describes a control specimen subjected to compression. FIG. 2b describes a test specimen of chusca to which the solution under study was applied and subjected to analysis after 7 days. FIG. 2c describes a test specimen of chusca, to which the solution under study was applied and subjected to analysis after 14 days. FIGS. 2a, 2b, 2c show the result of applying compression, as shown in the graph of FIG. 1.

FIG. 3 describes a graph of the sample of fine material (chusca) obtained in the mine. The graph describes the particle size distribution of the sample.

FIGS. 4a, 4b show treatment of chusca with the solution under study. FIG. 4a is the control, i.e., chusca treated with water. FIG. 4b describes Chusca treated with the solution under study. The red circles highlight the larger pieces of material that were obtained only by testing in the presence of the solution under study.

FIG. 5 describes a strength comparison between a control tailings test specimen (treated with water) and a tailings test specimen treated with the solution under study for 7 days.

FIG. 6a describes the control tailings specimen and FIG. 6b describes the specimen treated with the solution under study after 7 days of treatment. FIGS. 6a and 6b show the specimens after applying the compression described in the graph of FIG. 5.

### DESCRIPTION OF THE INVENTION

This patent application describes a composition, a process and its use, which removes silica present in suspended particulate matter in the environment. It also eliminates the resuspension of silica from the original emission spot by turning silica into heavier and more stable minerals, by creating new compositions in the soil such as gehlenite, calcite, gypsum, anorthite, kaolinite, muscovite, orthoclase, among others. All of these have a non-breathable size.

This patent application describes cementation of material by using soil particulate matter, using bacteria capable to generate CaCO3, applying a process known as “Microorganism Induced Carbonate Precipitation” (MICP). A product developed by Domolif, based on biomineralizing bacteria is used. It has been found that the application of MICP provides cohesion and increases stiffness of granular materials, as well as sequestering heavy metals.

In one embodiment of this invention, the biotechnological composition includes microorganisms, more specifically bacteria. In one embodiment of this invention, the bacteria are selected from the families Bacillaceae, Caryophanaceae, Pseudomonadaceae, Comamonadaceae, Lactobacilloeae, Micrococcaceae, Halomonadaceae, Halomonadaceae and Myxococcaceae, more specifically, the bacteria are selected from the genera Bacillus, Sporosorcina, Pseudomonas, Variovorax, Leuconostoc, Micrococcus, Deleya, Halomonas, Myxococcus. In one embodiment of this invention, the bacteria used for cementation are selected from among the following species Bacillus subtilis, Sporosarcina pasteurii, Pseudomonas sp., Variovorax sp., Leuconostoc mesenteroides, Micrococcus sp., Deleya halophila, Halomonas eurihalina and Myxococcus xanthus.

In one embodiment of this invention, the biotechnological composition includes a genetically modified bacterium that possesses a natural biocementation activity and synthesizes an enzyme capable of transforming silica dioxide by means of a biological activity known as silicification, aimed to reducing the risk of silicosis caused by the inhalation of crystalline SiO2. Silica (SiO2) has a molecular weight of 60 g/mol and 150 nm in diameter which can be turned, among other minerals, into gehlenite (Ca2Al(SiAlAl)O7) which has a molecular weight of 274.2 g/mol. When it binds to other molecules it can reach sizes greater than 10 μm. This size change turns gehlenite into a NON-breathable molecule. Therefore, this biotechnological composition is useful to reduce—in situ—the silica present in environmental particulate matter in suspension, thus comprising:
Bacteria having natural biocementation activity, where the bacteria are present in a concentration between 10⁶-10⁸ bacteria/mL;
Bacteria with a silicification activity, where the bacteria are present in a concentration between 10⁶-10⁸ bacteria/mL;
Culture medium, which may contain in its formula: yeast extract 5 g/L, casein peptone or soybean peptone 5-10 g/L, sodium chloride 5 g/L.
Calcium media, consisting of yeast extract (between 1-5 g/L), a source of sugars, which may be glucose, lactose or sugar sources such as whey and a source of calcium such as calcium chloride or other calcium salt.
Optionally, the bacteria can be in a solution or dry, in this case it will also have the additive maltodextrin or a similar one.

In one embodiment of this invention, the biotechnological composition generates minerals, based on quartz (SiO2) in the source of origin (soil, tailings or other materials), known as: gehlenite (Ca₂Al(SiAl)O₇), Anorthite (CaAl₂Si₂O₈), Kaolinite (Al₂Si₂O₅(OH)₄), Muscovite-3T (K,Na) (Al, Mg,Fe)₂ (Si₃·1Al0.9)O₁₀ (OH)₂), Orthoclase (KAlSi₃O₈). In one embodiment of this invention, the genetically modified bacterium with natural biocementation activity is selected from the genus Bacillus and generates calcite as a product (CaCO₃). The natural biocementation activity is not performed by the urease enzyme, using another metabolic pathway for the transformation of the source from calcium to calcium carbonate, therefore, this microorganism does not require urea for its cementation activity, unlike the bacteria reported in the dust control patent CL 51248.

In one embodiment of this invention, the enzyme synthesized by the genetically modified bacteria corresponds to bioremediase, which leaches silicon dioxide from the soil, thus turning it into silicon hydroxide or (water soluble) salicylic acid. Finally, in the presence of calcium and aluminum (elements naturally found as salts in soil or tailings), this compound forms precipitates such as gehlenite (Ca2Al(SiAl)O7) and other crystalline forms. In one embodiment of this invention, said bacterium contains the gene inserted in its genome and constitutively expresses the enzyme, transformed by methods known to the person skilled in the art.

In one embodiment of this invention, bacteria are in a quantity between 10⁶-10⁸ bacteria/mL.

In one embodiment of this invention, the biotechnological composition includes calcium salts to form calcite. In one embodiment of this invention, the biotechnological composition includes culture media known to the person skilled in the art.

In one embodiment of this invention, the process comprises applying the composition of the present patent application, wherein said composition comprising a bacterium is applied by directly spraying, sprinkling, dripping or mixing onto the surface containing such particulate matter. Within a few hours, a high percentage of SiO2 turns into gehlenite and/or other silicon minerals, becoming contained in a matrix that also helps to suppress particulate matter due to its new structure. In one embodiment of this invention, the applied sprayed composition can vary between 2-5 L/m²

In one embodiment of this invention, use of the composition of this patent application is described, and is aimed to contain and reduce—in situ—silica present in suspended particulate matter found in soil, tailings or other similar materials that need to be controlled in the environment, such as in mining operations, especially on mine pit roads, crushing areas and loading faces, as well as in tailing dams.

### EXAMPLES

Gehlenite formation has been linked to and proven to increase compressive strength.

The application of the treatment to the material received with a biological additive is shown below. This treatment was applied within the compaction humidity of the material, defined for this case as ω=15-20%. The compacted soil/tailings samples were stored in an incubator at a temperature of 28 °C, keeping constant humidity conditions. After 7 and 14 days of treatment, the respective test specimen was dried at room temperature until it reached a constant weight. Finally, the simple compression test was performed on each test specimen and these were compared with the control test specimens.

### Example 1

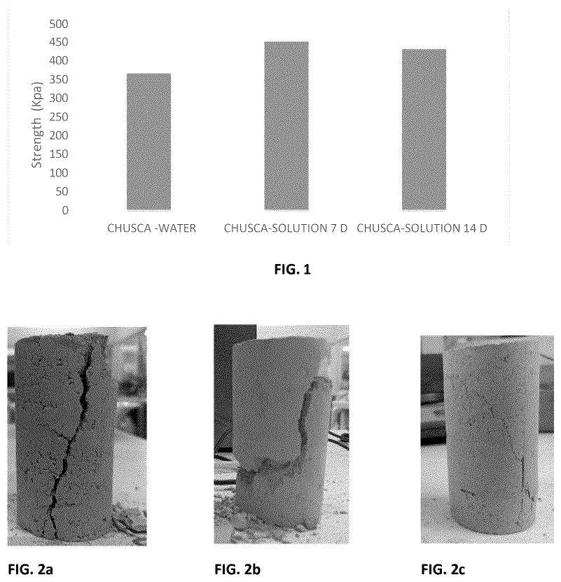

In another test, particulate soil material with a particle size of 90%<0075 (mm) is used. This material is known as “chusca” (fine dust). The results are shown in FIG. 1 and summarized in Table #2.

Particulate soil matter or chusca is used. Unconfined compressive strength tests were performed on samples as shown in FIGS. 2a, 2b, 2c.

Then, the compositional treatment was applied to three (3) test specimens within the compaction humidity. They were stored at controlled temperature conditions and the medium humidity was kept constant. After seven (7) days, the test specimens were dried at ambient conditions until they reached a condition of residual moisture, i.e., the mass of the material remained constant for two consecutive days. The test specimens were tested once a constant humidity condition was reached, thus obtaining the results shown in FIG. 1.

Additionally, the same treatment process was performed on three (3) other test specimens, but these were stored under controlled conditions for 14 days. Afterwards, we waited till obtaining a constant humidity condition, in order to perform the simple compression test also shown in FIG. 1.

The test specimens obtained when compression was applied are shown in FIGS. 2a, 2b, 2c. FIG. 2a is the control test specimen. FIG. 2b is the test specimen with the solution under study applied and analyzed on day 7 (7 D). Test specimen 2c is the test specimen with the solution under study, analyzed after 14 days (14 D).

In this test, fine particulate matter from the soil is used and it is observed that the test specimens show a significant difference in strength, observable to the naked eye. That is, when the composition under study is applied, better strength values are obtained in the specimen. This scenario was expected, since when applying the solution under study results in the cementation of the chusca, thus obtaining a stronger test specimen.

TABLE # 1
Strength applied to specimens shown in FIG. # 1

| | STRENGTH | Sample |
|---|---|---|
| SAMPLE | q(Kpa) | granulometry |
| CHUSCA -WATER | 365 | 90% < 0075(mm) |
| CHUSCA-SOLUTION 7 D | 450 | |
| CHUSCA-SOLUTION 14 D | 430 | |

FIG. 3 displays a graph showing the granulometry of the fine material obtained at the mine.

### Example 2

A biotechnological product is provided that uses a bacterium to turn SiO2 into a stable material named as gehlenite.

Gehlenite is larger in size. It eliminates resuspension and lowers the possibility of being inhaled by the worker, thus reducing the risk of silicosis in workers.

High silica and mining soil mixtures are tested with microorganisms with silicification activity.

The product is tested on 10 m² of mining soil. Post-application samples are made and SiO2 and gehlenite measurements are taken before and after treatment.

A lab test was performed with armed test specimens by mixing 50 grams of chusca with 10 ml of the culture for the lab tests. The control samples were tested with distilled water or sterile culture medium. It was incubated for 1 day at 37 °C. Then inoculations were continued aimed to increase the content of modified bacteria in the material.

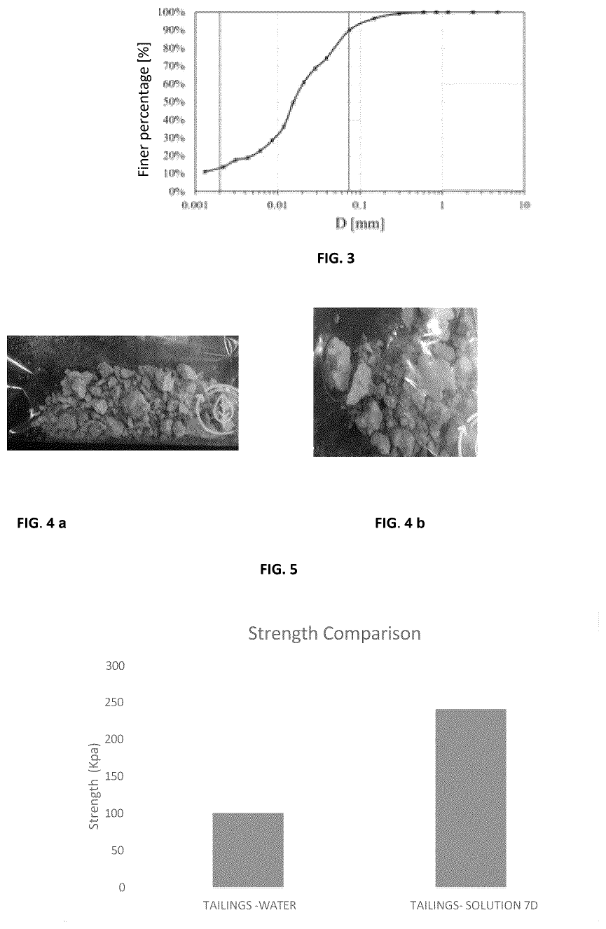

FIG. 4 describes photographs of the samples result after treatment with the transformed bacteria. FIG. 4a is a control sample, in the absence of bacteria. FIG. 4b is a sample in the presence of bacteria.

In the presence of bacteria, the formation of large pieces of material produced by the action of the modified bacteria can be seen (red circles).

The results of the analysis are as follows.

Results of Table #3: The content of elements present in the chusca was analyzed, giving as main result the presence of 48% of SiO2 or silica in the sample.

TABLE # 3
Initial analysis of chusca. Elements expressed as a percentage of the total

| SiO2 | TiO2 | Al2O3 | Fe2O3 | MnO | MgO | CaO | Na2O | K2O | P2O5 | ppc (%) | H2O· (%) |
|---|---|---|---|---|---|---|---|---|---|---|---|
| 48.1 | 0.5 | 22.5 | 4.8 | 0.0 | 0.9 | 1.6 | 0.8 | 3.7 | 0.2 | 11.6 | 0.9 |

The following table describes the results of the chusca after applying the treated enzyme.

TABLE # 4
Bacteria-treated chusca. Elements expressed
in percentage of the total

| Elements | Formula | % Total |
|---|---|---|
| Quartz | SiO₂ | 28.6 |
| Anorthite | CaAl₂Si₂O₈ | 11.3 |
| Boehmite | AlO(OH) | 4.0 |
| Calcite | Ca(CO₃) | 4.7 |
| Gehlenite | Cₐ₂Al(Al,Si)₂O₇ | 3.4 |
| Gypsum | CaSO₄•2H₂O | 3.8 |
| Kaolinite | Al₂Si₂O₅(OH)₄ | 12.3 |
| Muscovite-3T | (K,Na)(Al,Mg,Fe)₂(Si_{3.1}Al_{0.9})O₁₀(OH)₂ | 8.6 |
| Orthoclase | KAlSi₃O₈ | 18.6 |
| Pyrite | FeS₂ | 4.8 |

### CONCLUSIONS

The main result was proving that chusca treated with the transformed bacteria caused the formation of gehlenite (3.4%). This is a robust result because a bacterium that has no natural silicification activity was used and the gene was inserted into it. Therefore, the conclusion is that the activity of the treated enzyme is the cause of gehlenite formation in the chusca sample.
Approximately 20% decrease in silica is observed by correlating the original sample and the treated sample: 48.1% to 28.6%.
In addition to the formation of gehlenite, the formation of several crystals larger than silica was observed, such as calcite, gypsum, anorthite, kaolinite, muscovite, orthoclase. The last 4 also have silica in their structure. This result is very promising as the modified bacterium uses the silicon source to generate not only gehlenite, but also several crystals that are made up of silica. This is in total, 54.2% of the sample. This helps to decrease silica concentration (SiO2) which is the cause of silicosis due to its smaller particle size. That is to say, it ranges from 48% silica in the sample to 54.2% of larger minerals, whose structure is made up of silica.

### Example 3

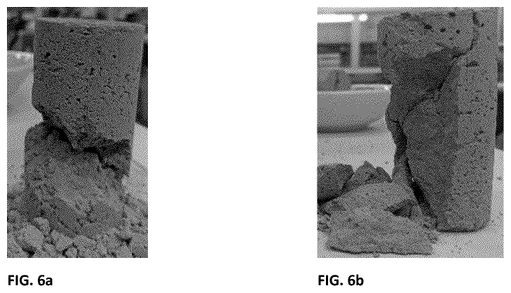

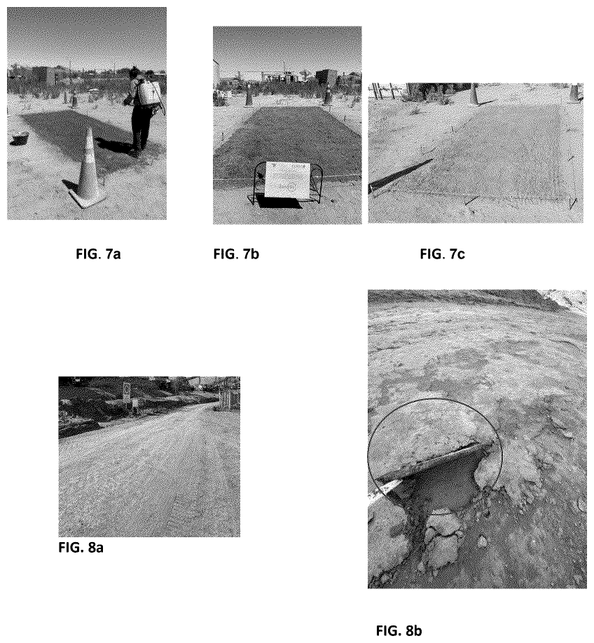

Tailings sample with a particle size of 62%<0.075 (mm) is used. The results obtained are shown in FIG. 5. FIGS. 6a and 6b show the test specimens after compression has been applied. The test specimen #6b was analyzed after 7 days and better results were obtained, as shown in Table #3 and the graph shown in FIG. 5.

It is clear that by using the composition under study there is an improvement in the compression results, thus improving the strength of the test specimen. This indicates that cementation is higher when the composition under study is used.

TABLE # 5
Applied strength of test specimens shown in FIG. # 1

| | STRENGTH | Sample |
|---|---|---|
| SAMPLE | q(Kpa) | granulometry |
| TAILING -WATER | 100 | 62% < 0.075 (mm) |
| TAILING - SOLUTION 7 D | 240 | |

The treatment was applied within the compaction humidity (15-20%) at dry density d=1.56 g/cm³. The untreated material was compacted, then left to dry at room temperature until it reached residual moisture, i.e., the mass remained constant for two consecutive days. Then, a simple compression test was performed, thus obtaining the results shown in FIG. 5.

Additionally, two (2) test specimens were tested and stored in curing conditions for 7 days. They were then dried until reaching the residual moisture content and were tested to determine their simple compression as shown in FIGS. 6a and 6b.

In the cured tailings, a higher strength than the control material can be clearly observed.

### Example 4

### Product Testing in Mining Soil.

The presence of Gehlenite is determined indirectly by compressive strength testing.

Below is the test of the biotechnological product (genetically modified bacteria) in mining soil.

FIG. 7a. describes soil application of the product by spraying (2-5 L/m²). FIG. 7b. describes the application area completely treated with the product. After 7 days, the condition of the test area was observed. The surface was found to be completely hard (FIG. 7 c), similar to what was observed in the test specimen test (FIG. 7 d).

FIG. 8a. describes a second test area, a road completely treated with the product (3-5 L/m²)

After 7 days, the condition of the test area was examined and a hard crust was formed (FIG. 8b, red circle), similar to what was observed in the test specimens test (FIG. 8b, red circle).

These results suggest that gehlenite formation was obtained in the field and most probably that of other larger sized species made up of silica.

### GENERAL CONCLUSIONS OF THE SOIL TESTS

In the tests, the treated surface showed an evident change in hardness after being treated with the modified bacteria, thus proving the formation of larger elements such as gehlenite.

While this invention has been described under the modalities indicated above, it might seem apparent that other alternatives, modifications or variations would deliver the same results. However, we have been able to determine that the subject described in this patent application is fundamental to the success of the invention described herein. Accordingly, the embodiments of the invention are intended to be illustrative, not limiting. Various changes can be made without deviating from the spirit and scope of the invention as defined in the following claims.

All patents, patent applications, scientific articles and other public documents that to the best of the applicant's knowledge constitute the state-of-the-art, have been adequately cited in this patent application.

1. A biotechnological composition to reduce—in situ—silica present in suspended particulate matter in the environment, wherein comprises:
bacteria that have natural biocementation activity, where the bacteria are present in a concentration between 10⁶-10⁸ bacteria/mL;
bacteria that have silicification activity, where the bacteria are present in a concentration between 10⁶-10⁸ bacteria/mL; and culture media and drying stabilizers, preferably maltodextrin.

2. The composition described in claim 1, wherein the culture medium comprises yeast extract 5 g/L, casein or soy peptone 5-10 g/L or sodium chloride 5 g/L.

3. The composition described in claim 1, wherein that bacteria can be genetically modified, and more particularly, genetically modified bacteria have a natural biocementation activity which is selected from the genus Bacillus thus generating calcite as a product. (CaCO₃).

4. The composition described in claim 1, wherein the enzyme synthesized by the genetically modified bacteria has silicification activity.

5. The composition described in claim 1, wherein said bacterium contains the gene inserted in its genome and constitutively expresses the enzyme.

6. The composition described in claim 1, wherein said composition is applied directly by spraying, sprinkling, dripping or mixing onto the surface containing particulate matter.

7. The composition described in claim 1, wherein the composition is applied directly by spraying.

8. The composition described in claim 1, wherein the dripped composition applied can vary between 2-5 L/m².

9. The composition described in claim 1, wherein the bacteria are selected from the families Bacillaceae, Caryophanaceae, Pseudomonadaceae, Comamonadaceae, Lactobacilloae, Micrococcaceae, Halomonadaceae, Halomonadaceae and Myxococcaceae, more specifically, the bacteria are selected from the genera Bacillus, Sporosorcina, Pseudomonas, Variovorax, Leuconostoc, Micrococcus, Deleya, Halomonas, Myxococcus.

10. A process to reduce—in situ—the silica present in suspended particulate matter in the environment, wherein comprises
Apply an effective amount of the biotechnological composition directly onto the environmental sources of suspended particulate matter.

11. The process described in claim 10, wherein is applied directly by spraying, sprinkling, dripping or mixing onto the surface containing particulate matter.

12. The process described in claim 10, wherein the direct application by spraying the applied composition can vary between 2-5 L/m².

13. Use of the biotechnological composition according to claim 1, wherein is useful to reduce—in situ—the silica present in suspended particulate matter in the environment, aimed to reducing the risk of silicosis.
